(19) United States
(12) Patent Application Publication
(10) Pub. No.: US 2022/0401011 A1
(43) Pub. Date: Dec. 22, 2022

(54) INFORMATION PROCESSING APPARATUS, INFORMATION PROCESSING METHOD, AND PROGRAM

(71) Applicant: SONY GROUP CORPORATION, TOKYO (JP)

(72) Inventor: YASUHIDE HYODO, TOKYO (JP)

(21) Appl. No.: 17/756,270

(22) PCT Filed: Nov. 10, 2020
(86) PCT No.: PCT/JP2020/041894

§ 371 Date: May 20, 2022

(30) Foreign Application Priority Data
Nov. 29, 2019 (JP) 2019-216827

(51) Int. Cl. A61B 5/00; A61B 5/16

(52) U.S. Cl. CPC A61B 5/4266; A61B 5/165

(57) ABSTRACT

[Object] To provide a technology capable of accurately inferring emotions of a living body.

[Solving Means] An information processing apparatus according to the present technology includes: a control unit. The control unit separates a perspiration signal into a first fluctuation component and a second fluctuation component and corrects the second fluctuation component on the basis of the first fluctuation component.

### TECHNICAL FIELD

The present technology relates a technology for processing a perspiration signal such as a skin conductance signal.

### BACKGROUND ART

In recent years, various measurement technologies for determining the emotions (psychological state) of a living body have been studied. When the emotions of the living body change, a signal is transmitted from the brain to the respective parts of the human body via the autonomic nervous system. For example, a change occurs in the respective functions of the heart, respiration, perspiration, skin temperature, vascular activity, and the like. Among these, mental perspiration is known as a physiological reaction representing the psychological state of arousal. It is known that the mental perspiration can be detected as a change in the value of a skin conductance signal or a skin impedance signal detected by an electrode in contact with a detection site.

For example, the following Patent Literature 1, Patent Literature 2, and Non-Patent Literature 1 describe a technology for detecting mental perspiration by a change in a skin conductance signal or a skin impedance signal.

It is known that the skin conductance signal is a combination of a skin conductance level (SCL) indicating a gradual change in perspiration on the skin surface and a skin conductance response (SCR) indicating an instantaneous change in perspiration.

In the past, the physiological mechanism of SCL/SCR observation in mental perspiration has been studied. In accordance with the following Non-Patent Literature 2, the dependency of SCR on SCL is represented by the following formula (1) using an equivalent circuit model.

dG={G₁²/(G₁+G₂+y)²}dy (1)

Note that in the formula (1), dG, G₁, G₂, and dy respectively represent a change in conductance (observed as SCR), a conductance of the dermis, a conductance of the stratum corneum, and a conductance of sweat gland activity. In this way, the SCL/SCR observation can be represented by the equivalent circuit model of the stratum corneum, the dermis, sweat gland activity.

### CITATION LIST

### Patent Literature

Patent Literature 1: Japanese Unexamined Patent Application Publication No. 2016-516461
Patent Literature 2: Japanese Patent Application Laid-open No. 1998-254613
Non-Patent Literature 1 Jain, Swayambhoo, et al, “A compressed sensing based decomposition of electrodermal activity signals,” IEEE Transactions on Biomedical Engineering 64.9 (2017): 2142-2151.
Non-Patent Literature 2 Boucsein, Wolfram, “Electrodermal activity”, Springer Science & Business Media, 2012.

### DISCLOSURE OF INVENTION

### Technical Problem

In such a field, a technology capable of accurately inferring emotions of a living body is desired.

In view of the circumstances as described above, it is an object of the present technology to provide a technology capable of accurately inferring emotions of a living body.

### Solution to Problem

An information processing apparatus according to the present technology includes: a control unit. The control unit separates a perspiration signal into a first fluctuation component and a second fluctuation component and corrects the second fluctuation component on the basis of the first fluctuation component.

By inferring emotions of a living body on the basis of the second fluctuation component corrected in this way, it is possible to accurately infer the emotions of the living body.

In the information processing apparatus, the control unit may correct the second fluctuation component by a gain relating to the first fluctuation component.

In the information processing apparatus, the gain may be a value that monotonically decreases with respect to a value of the first fluctuation component.

In the information processing apparatus, the gain may be a value that monotonically decreases with respect to a value of the first fluctuation component with respect to a first reference value.

In the information processing apparatus, the control unit may determine whether or not emotions are in a physiologically quiet state on the basis of a signal relating to the perspiration signal. In this case, the first reference value may be the first fluctuation component in a case where the emotions are in the physiologically quiet state.

In the information processing apparatus, the control unit may determine whether or not an activity state is a quiet state on the basis of at least one of a body motion signal based on a body motion change or a pressure signal based on a pressure change with skin. In this case, the first reference value may be the first fluctuation component in a case where the activity state is the quiet state and the emotions are in the physiologically quiet state.

In the information processing apparatus, the control unit may correct a value of the second fluctuation component with respect to a second reference value.

In the information processing apparatus, in a case where the control unit determines whether or not the emotions are in the physiologically quiet state on the basis of a signal relating to the perspiration signal, the second reference value may be the second fluctuation component in a case where the emotions are in the physiologically quiet state.

In the information processing apparatus, in a case where the control unit determines whether or not the activity state is the quiet state on the basis of at least one of the body motion signal based on the body motion change or the pressure signal based on the pressure change with the skin, the second reference value may be the second fluctuation component in a case where the activity state is the quiet state and the emotions are in the physiologically quiet state.

In the information processing apparatus, the first fluctuation component may be a baseline fluctuation component of the perspiration signal.

In the information processing apparatus, the second fluctuation component may be an instantaneous fluctuation component of the perspiration signal.

An information processing method according to the present technology includes: separating a perspiration signal into a first fluctuation component and a second fluctuation component; and correcting the second fluctuation component on the basis of the first fluctuation component.

A program according to the present technology causes a computer to execute the following processing of: separating a perspiration signal into a first fluctuation component and a second fluctuation component; and correcting the second fluctuation component on the basis of the first fluctuation component.

### BRIEF DESCRIPTION OF DRAWINGS

FIG. 1 is an external view showing a wearable device according to a first embodiment.

FIG. 2 is a block diagram showing an electrical configuration of the wearable device according to the first embodiment.

FIG. 3 is a perspective view of a band as viewed from the back side.

FIG. 4 is a cross-sectional view taken along the line A-A′ shown in FIG. 3.

FIG. 5 is a diagram showing a state when a deformable member is disposed between a perspiration sensor and a pressure sensor.

FIG. 6 is a diagram showing a specific configuration of part of a control unit.

FIG. 7 is a diagram showing an example of the relationship between a gain and dSCL.

FIG. 8 is a diagram showing the relationship between SCL and SCR (dSCL and dSCR) with a finger for each experimental task.

FIG. 9 is a diagram showing the relationship between SCL and SCR (dSCL and dSCR) with a wrist for each experimental task.

FIG. 10 is a diagram comparing the relationship between SCR without correction and SCR with correction in the case where the measurement target is the wrist.

### MODE(S) FOR CARRYING OUT THE INVENTION

### First Embodiment

<Configuration of Entire Wearable Device and Configurations of Respective Units>

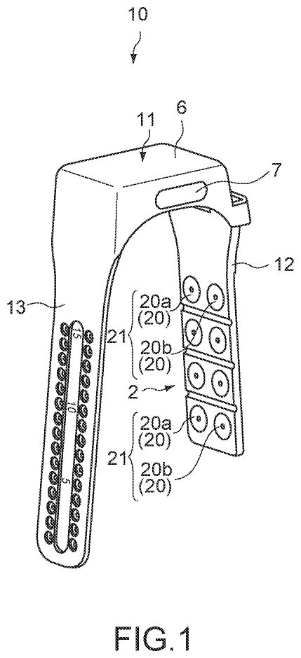

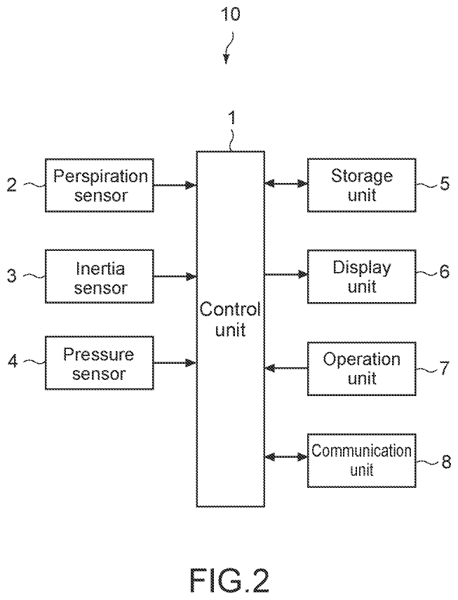

Hereinafter, an embodiment according to the present technology will be described with reference to the drawings. FIG. 1 is an external view showing a wearable device 10 according to a first embodiment. FIG. 2 is a block diagram showing an electrical configuration of the wearable device 10 according to the first embodiment.

This wearable device 10 (example of the information processing apparatus) is of a wristwatch type (wristband type) and is used by being wrapped around a user's wrist.

As shown in FIG. 1 and FIG. 2, the wearable device 10 includes a case 11 and two bands 12 and 13 provided on both ends of the case 11. Further, the wearable device 10 includes a control unit 1, a perspiration sensor 2, an inertia sensor 3, a pressure sensor 4, a storage unit 5, a display unit 6, an operation unit 7, and a communication unit 8.

The case 11 has a rectangular parallelepiped shape having a thin thickness, and is formed of, for example, a material such as a metal and a resin. The display unit 6 is provided on the upper surface of the case 11, and the operation unit 7 is provided on the side surface of the case 11. Further, the control unit 1, the inertia sensor 3, the storage unit 5, the communication unit 8, and the like are modularized and built in the case 11.

The display unit 6 includes, for example, a liquid crystal display or an EL display (EL: Electro Luminescence). The display unit 6 displays, for example, the current time, icons indicating various applications such as music, games, mails, and browsers, or various images such as moving images and still images by executing applications, in accordance with the control of the control unit 1.

The operation unit 7 is, for example, an operation unit of various types such as a pressing type and a proximity type, and detects an operation by a user and outputs the operation to the control unit 1. Note that the operation unit 7 may include a proximity sensor provided on the display unit 6.

The bands 12 and 13 each have a shape that is thin and long in one direction. The bands 12 and 13 are formed of, for example, a material such as rubber, leather, and an organic resin so that the bands 12 and 13 can be easily wrapped around the user's wrist and easily come into contact with the wrist (skin).

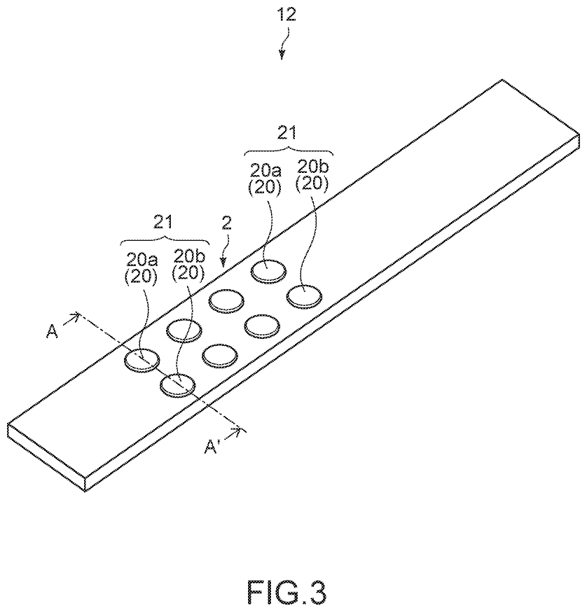

FIG. 3 is a perspective view of the band 12 as viewed from the back side. As shown in FIG. 1 and FIG. 3, a plurality of electrode pairs 21 in the perspiration sensor 2 is provided on the back side (side in contact with the wrist) of the band 12. The electrode pairs 21 in the perspiration sensor 2 each include a first electrode 20a and a second electrode 20b. The plurality of electrode pairs 21 is arranged at equal intervals along the length direction of the band 12, and the first electrodes 20a and the second electrodes 20b in the electrode pairs 21 are arranged along the width direction.

The electrode pair 21 of the perspiration sensor 2 is provided on the band 12 so as to be exposed from the back side of the band 12 so that the electrode pair 21 easily comes into contact with the user's wrist (skin) when the band 12 is wrapped around the wrist. In the examples shown in FIG. 1 and FIG. 3, the shape of the electrode 20 (general term of the first electrode 20a and the second electrode 20b) is a circular shape, but this shape may be a polygon such as a triangle and a square. The shape of the electrode 20 is not particularly limited.

The perspiration sensor 2 is a sensor that detects a skin conductance signal (perspiration signal) based on emotions of a living body and is capable of outputting the skin conductance signal to the control unit 1. The perspiration sensor 2 is in contact with the user's skin and detects sweat secreted from a sweat gland (e.g., eccrine gland) of the skin. The perspiration sensor 2 is capable of detecting, as electro dermal activity (EDA), a change in the ease of passage of a current of the skin (skin conductance) based on perspiration.

The perspiration sensor 2 includes the electrode pairs 21 including the first electrodes 20a and the second electrodes 20b, a voltage/power source unit that generates a potential difference between the first electrode 20a and the second electrode 20b, a detection unit that detects a current flowing between the first electrode 20a and the second electrode 20b, and the like. Further, the perspiration sensor 2 includes a current/voltage conversion unit that converts the detected current into a voltage, an amplification unit that amplifies a skin conductance signal, a filter processing unit that filters the amplified signal, and the like.

The voltage to be applied between the first electrode 20a and the second electrode 20b may be an alternating current or a direct current. In this embodiment, a skin conductance signal is used as a perspiration signal. Instead of the skin conductance signal, a skin impedance signal (perspiration signal), a skin resistance signal (perspiration signal), or the like may be used.

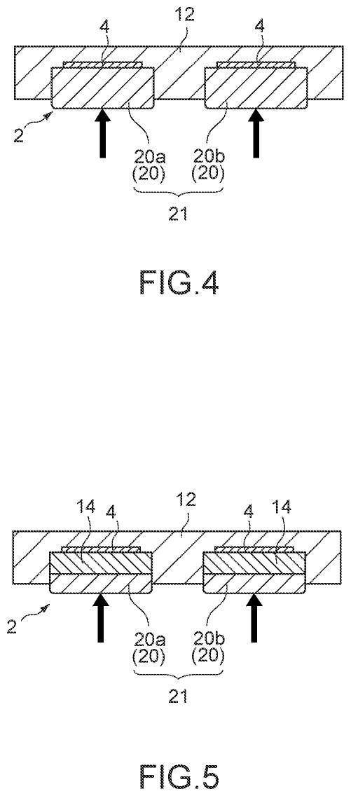

FIG. 4 is a cross-sectional view taken along the line A-A′ shown in FIG. 3. As shown in FIG. 4, in the band 12, the pressure sensor 4 is provided on the upper side of the electrode 20 of the perspiration sensor 2 (on the side opposite to the skin side with respect to the electrode 20). That is, the perspiration sensor 2 (electrode 20), the pressure sensor 4, and the band 12 are stacked in this order from the skin side at corresponding locations to achieve a three-layer structure.

The region in which the pressure sensor 4 is disposed overlaps with the region in which the perspiration sensor 2 (electrode 20) is disposed, and the pressure sensor 4 is disposed on the side opposite to the skin side with respect to the perspiration sensor 2 and directly above the perspiration sensor 2 (electrode 20). Although the pressure sensor 4 overlaps with the perspiration sensor 2 (electrode 20) in all regions in the example shown in FIG. 4, the pressure sensor 4 may overlap with the perspiration sensor 2 in a partial region.

The pressure sensor 4 is a sensor that detects a pressure signal based on a pressure change with skin (see black arrows in FIG. 4) and is capable of outputting the detected pressure signal to the control unit 1. The pressure sensor 4 includes, for example, a device whose voltage, current, resistance, or the like changes depending on the pressure (e.g., a piezoelectric device or a pressure-sensitive conductive elastomer). Typically, as the pressure sensor 4, any sensor may be used as long as it is capable of detecting pressure.

Note that a deformable member 14 may be disposed between the perspiration sensor 2 (electrode 20) and the pressure sensor 4. FIG. 5 is a diagram showing a state when the deformable member 14 is disposed between the perspiration sensor 2 and the pressure sensor 4.

In the example shown in FIG. 5, the perspiration sensor 2 (electrode 20), the deformable member 14, the pressure sensor 4, and the band 12 are stacked in this order from the skin side at corresponding locations to achieve a four-layer structure. The deformable member 14 is deformed by, for example, pressure, and can be restored to the original shape by releasing the pressure. Examples of the material used for the deformable member 14 include various types of rubber such as silicon rubber and organic resins. Typically, the deformable member 14 is formed of a material that is deformed more than the band 12 when pressed at the same pressure.

In the case where the deformable member 14 is provided, the deformable member 14 is compressed and deformed when the perspiration sensor 2 is pressed by the skin, and the force generated as the reaction force to this compression deformation is transmitted to the pressure sensor 4, whereby the pressure with the skin can be detected more appropriately.

With reference to FIG. 2 again, the inertia sensor 3 is a sensor that detects an inertial signal based on a body motion change (a body motion signal: acceleration signal, an angular velocity signal, or the like), and is capable of outputting this inertial signal to the control unit 1. This inertia sensor 3 includes a 3-axis acceleration sensor that detects the acceleration in the 3-axis direction and an angular velocity sensor that detects angular velocity around the three axes.

In this embodiment, the detection axes of the inertia sensor 3 are three axes, but the detection axes may be one axis or two axes. Further, although two types of sensors are used as the inertia sensor 3 in this embodiment, one or three or more types of sensors may be used as the inertia sensor 3. Note that other examples of the inertia sensor 3 include a velocity sensor and an angle sensor.

The inertia sensor 3 and the pressure sensor 4 may be calibrated at a predetermined timing such as a timing when the user wore the wearable device 10.

The control unit 1 executes various operations on the basis of various programs stored in the storage unit 5 to integrally controls the respective units of the wearable device 10.

The control unit 1 is realized by hardware or a combination of hardware and software. The hardware is configured as part or all of the control unit 1. Examples of this hardware include a CPU (Central Processing Unit), a DSP (Digital Signal Processor), an FPGA (Field Programmable Gate Array), an ASIC (Application Specific Integrated Circuit), and a combination of two or more of these.

Note that the specific configuration and processing of the control unit 1 will be described below in detail.

The storage unit 5 includes a non-volatile memory for storing various programs necessary for the processing of the control unit 1 and various types of data, and a volatile memory used as a work area of the control unit 1.

The communication unit 8 is configured to be capable of communicating with a different device other than the wearable device 10. Examples of the different device capable of communicating with the wearable device 10 include various PCs such as desktop PCs (Personal Computers), mobile phones (including smartphones), and server apparatuses on the network.

<Method According to Present Technology>

Now, a method used in the present technology will be simply described before describing the specific configuration of the control unit 1.

Regarding stimulus for humans, there are a higher-order pathway in which the stimulus passes through the amygdala via the sensory thalamus and sensory cortex and a lower-order pathway in which the stimulus passes through the amygdala from the sensory thalamus. The stimulus is analyzed and transmitted to the amygdala in the higher-order pathway, which takes time, but the processing of the higher-order cerebral cortex is omitted in the lower-order pathway, which makes it possible to rapidly evaluate the stimulus. It is known that the amygdala causes physical reactions such as emotional reactions, autonomic reactions, and hormone secretion through the hypothalamus and autonomic nerves. The sweat glands under the skin are connected to the autonomic nerves and perspire in response to stimuli.

The perspiration is roughly classified into thermal perspiration for regulating body temperature in a hot environment or when exercising, mental perspiration when receiving mental stimuli such as mental tension and emotional changes, gustatory perspiration when eating spicy or irritating foods, and the like.

Incidentally, it is said that the sweat glands with a lot of mental perspiration that is an emotional reaction have limited positions, are often found on the fingertips, palms, soles, and the like, and less found on other locations such as a wrist position. The fingertips, palms, and soles are appropriate for measuring mental perspiration, but the burden on a subject is heavy because the behavior in daily life is restricted. Meanwhile, the wrist position or the like does not easily affect the behavior in daily life. In this respect, the wrist position or the like is suitable for measuring perspiration.

However, since the wrist position or the like is a location with a few sweat gland, the value of a skin conductance signal based on mental perspiration is weak and there is a possibility that mental perspiration cannot be appropriately detected.

As described above, the skin conductance signal is a combination of a skin conductance level (hereinafter, abbreviated as SCL) indicating a gradual change in perspiration on the skin surface and a skin conductance response (hereinafter, abbreviated as SCR) indicating an instantaneous change in perspiration. Therefore, two signals, SCL and SCR, can be separated from the skin conductance signal.

Of these SCL and SCR, SCR indicating an instantaneous change in perspiration appropriately represents emotions (psychological state) of a user by mental perspiration. Therefore, it is conceivable that if SCR can be accurately detected, the current emotions of the user can be accurately determined.

Meanwhile, SCR cannot be detected with high sensitivity in the portion with a few sweat gland, such as the wrist portion. In particular, the portion with a few sweat gland, such as the wrist portion, has a problem that the rise of SCR is slow. This is a constraint in the case where, for example, information regarding emotions of a user in real time is used for various applications such as games.

Here, the formula (1) disclosed in the above-mentioned Non-Patent Literature 2 is described again.

dG={G₁²/(G₁+G₂+y)²}dy (1)

In the formula (1), dG, G₁, G₂, and dy respectively represent a change in conductance (observed as SCR), a conductance of the dermis, a conductance of the stratum corneum, and a conductance of sweat gland activity.

As is clear from the formula (1), SCR has a dependency on SCL. In the present technology, a method of improving the detection sensitivity of SCR by correcting SCR on the basis of SCL using the dependency of SCR on SCL is adopted.

In the equivalent circuit model according to the formula (1), it is difficult to directly measure the conductance of the dermis, the conductance of the stratum corneum, and the conductance of sweat gland activity, and the conductance value to be observed depends on the device characteristics such as the type of the electrode 20. For this reason, it is difficult to directly correct SCR by the equivalent circuit model according to the formula (1) (that is, in the present technology, the formula (1) is not directly used).

For this reason, in the present technology, SCR is corrected by a correction model established in advance (look-up table described below).

As can be seen from the description here, the present technology is a technology particularly useful, for example, in the case where perspiration is detected in a location with a few sweat gland, such as the wrist position. However, this does not mean that the present technology is limited to the application in which perspiration is detected in a location with a few sweat gland, such as the wrist position. That is, the present technology can be used for any part of the body in humans (or animals) for the purpose of perspiration in the skin regardless of the number of sweat glands.

<Specific Configuration of Control Unit 1>

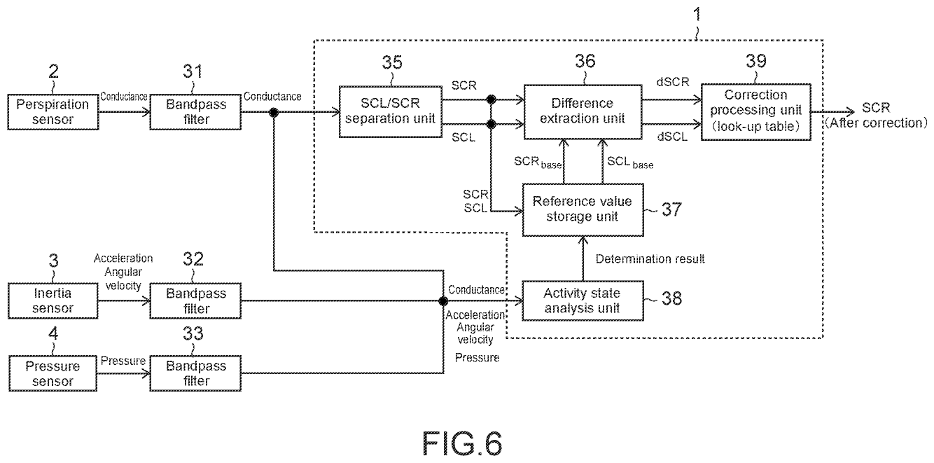

Next, a specific configuration of part of the control unit 1 will be described. FIG. 6 is a diagram showing a specific configuration of part of the control unit 1.

As shown in FIG. 6, the control unit 1 includes an SCL/SCR separation unit 35, a difference extraction unit 36, a reference value storage unit 37, an activity state analysis unit 38, and a correction processing unit 39.

(SCL/SCR Separation Unit 35)

A skin conductance signal, which is converted into a fluctuation component by passing through a bandpass filter 31, is input to the SCL/SCR separation unit 35. The SCL/SCR separation unit 35 is configured to be capable of separating the input skin conductance signal into an SCL signal and an SCR signal. Further, the SCL/SCR separation unit 35 is configured to be capable of outputting the separated SCL signal and SCR signal to the difference extraction unit 36 and the reference value storage unit 37.

SCL (first fluctuation component) is a low frequency component of a skin conductance signal and is a baseline fluctuation component. Further, SCR (second fluctuation component) is a high frequency component of a skin conductance signal and is an instantaneous fluctuation component.

In the signal separation, the SCL/SCR separation unit 35 calculates an SCR signal by, for example, extracting an SCL signal from a skin conductance signal by a smoothing filter and subtracting the extracted SCL signal from a skin conductance signal.

Alternatively, the SCL/SCR separation unit 35 may subtract an SCL signal from a skin conductance signal and then further extract an impulse rising component from the signal by a bi-exponential filter (since SCR has characteristics of rising quickly and falling slowly, this relationship is used). Then, the SCL/SCR separation unit 35 may obtain an SCR signal by smoothing the extracted signal by a smoothing filter.

(Activity State Analysis Unit 38)

A skin conductance signal, which is converted into a fluctuation component by passing through the bandpass filter 31, is input to the activity state analysis unit 38. Further, an inertial signal (an acceleration signal, an angular velocity signal), which is converted into a fluctuation component by passing through a bandpass filter 32, and a pressure signal converted into a fluctuation component by passing through a bandpass filter 33, are input to the activity state analysis unit 38.

The activity state analysis unit 38 determines, on the basis of the skin conductance signal, whether the situation in the contact state is the contact state or the non-contact state. The contact state is a state in which the perspiration sensor 2 (electrode pair 21) is in contact with the (living body), and the non-contact state is a state in which the perspiration sensor 2 (electrode pair 21) is not in contact with the skin.

Typically, the activity state analysis unit 38 compares the value of a skin conductance signal and a predetermined threshold value with each other, and determines, in the case where the value of the skin conductance signal is the threshold value or more, that the situation in the contact state is the contact state. Meanwhile, the activity state analysis unit 38 determines, in the case where the value of the skin conductance signal is less than the threshold value, that the situation in the contact state is the non-contact state. In the case where the situation in the contact state is the non-contact state, for example, a user may be notified of that the situation is the non-contact state, via the display unit 6.

Further, the activity state analysis unit 38 is configured to be capable of determining, on the basis of an inertial signal and a pressure signal, whether the situation in an activity state of a living body is an activity state, a quasi-activity state, or a quiet state. Note that regarding the situation in the activity state, the state other than the quiet state (the activity state and the quasi-activity state in this example) will be referred to as the non-quiet state.

Note that the activity state analysis unit 38 executes, in the case where the determination results of the contact/non-contact state is the contact state, this determination of an activity state. Meanwhile, the activity state analysis unit 38 typically does not execute, in the case where the determination result of the contact/non-contact state is the non-contact state, the determination of an activity state.

The activity state is, for example, a state in which the body and arms are moving a lot at the time of exercising, stretching, or the like. The quasi-activity state is, for example, a state in which some parts (fingers, wrists, etc.) of the body are moving small at the time of operating a smartphone, working on a PC, or the like. Further, the quiet state is, for example, a state in which the body is hardly moving at the time of sleep, naps, breaks, or the like.

The activity state analysis unit 38 determines, on the basis of an inertial signal, whether the situation in the activity state is the activity state or other states (the quasi-activity state, the quiet state). Typically, the activity state analysis unit 38 compares the value of an inertial signal and a predetermined threshold value with each other, and determines, in the case where the value of the inertial signal is a threshold value or more, that the situation is the activity state. Meanwhile, the activity state analysis unit 38 determines, in the case where the value of the inertial signal is less than the threshold value, the situation in the activity state is the state (the quasi-activity state and the quiet state) other than the activity state.

The activity state analysis unit 38 may be configured to be capable of executing norm value processing, buffering, maximum value filtering processing, and the like on the inertial signal. In this case, the activity state analysis unit 38 executes norm value processing on the inertial signal and buffers the inertial signal (acceleration norm, angular velocity norm, or the like) converted into a norm value.

Then, the activity state analysis unit 38 executes maximum value filtering processing on the norm value in the latest predetermined time, of the buffered norm values, to acquire the maximum value of the norm value. In this way, the activity state analysis unit 38 acquires the maximum value of the norm value at predetermined time intervals. The activity state analysis unit 38 compares the acquired maximum value of the norm value and a predetermined threshold value with each other to determine whether the situation in the activity state is the activity state of other states (the quasi-activity state, the quiet state).

Further, in the case where the situation in the activity state is the state other than the activity state, the activity state analysis unit 38 further determines, on the basis of a pressure signal, whether the situation in the activity state is the quasi-activity state or the quiet state. Typically, the activity state analysis unit 38 compares the value of the pressure signal and a predetermined threshold value with each other, and determines, in the case where the value of the pressure signal is the threshold value or more, that the situation is the quasi-activity state. Meanwhile, the activity state analysis unit 38 determines, in the case where the value of the pressure signal is less than the threshold value, that the situation is the quiet state.

The activity state analysis unit 38 may be configured to be capable of executing differential-absolute-value filtering processing, buffering, maximum value filtering processing, and the like on a pressure signal. In this case, the activity state analysis unit 38 executes differential absolute value processing on an inertial signal and buffers the pressure signal converted into a differential absolute value.

Then, the activity state analysis unit 38 executes maximum value filtering processing on the differential absolute value in the latest predetermined time, of the buffered differential absolute values to acquire the maximum value of the differential absolute value. In this way, the activity state analysis unit 38 acquires the maximum value of the differential absolute value in the pressure signal at predetermined time intervals. The activity state analysis unit 38 compares the acquired maximum value of the differential absolute value and a predetermined threshold value with each other to determine whether the situation in the activity state is the quasi-activity state or the quiet state.

The activity state analysis unit 38 is configured to determine the situation in the activity state and then output the result of the determination of the activity state to the reference value storage unit 37.

Note that in the description here, the case where the activity state analysis unit 38 determines, on the basis of both an inertial signal and a pressure signal, whether the situation in the activity state is the non-quiet state (the activity state, the quasi-activity state) or the quiet state has been described. Meanwhile, the activity state analysis unit 38 only needs to be configured to be capable of determining, typically on the basis of at least one of an inertial signal or a pressure signal, whether the situation in the activity state is the non-quiet state or the quiet state.

For example, the activity state analysis unit 38 may determine the non-quiet state and the quiet state on the basis of only the inertial signal, of the inertial signal and the pressure signal. In this case, the pressure sensor 4 can be omitted. In this case, for example, the activity state analysis unit 38 compares the value of the inertial signal and a predetermined threshold value with each other and determines, in the case where the value of the inertial signal is the threshold value or more, that the situation in the activity state is the non-quiet state. Meanwhile, the activity state analysis unit 38 determines, in the case where the value of the inertial signal is less than the threshold value, that the situation in the activity state is the quiet state.

Further, for example, the activity state analysis unit 38 may determine the non-quiet state and the quiet state on the basis of only the pressure signal, of the inertial signal and the pressure signal. In this case, the inertia sensor 3 can be omitted. In this case, for example, the activity state analysis unit 38 compares the value of the pressure signal and a predetermined threshold value with each other, and determines, in the case where the value of the pressure signal is the threshold value or more, that the situation in the activity state is the non-quiet state. Meanwhile, the activity state analysis unit 38 determines, in the case where the value of the pressure signal is less than the threshold value, that the situation in the activity state is the quiet state.

(Reference Value Storage Unit 37)

An SCL signal and an SCR signal are input from the SCL/SCR separation unit 35 to the reference value storage unit 37. Further, the result (the activity state, the quasi-activity state, or the quiet state) of determination in an activity state is input from the activity state analysis unit 38 to the reference value storage unit 37.

The reference value storage unit 37 is capable of updating and storing the SCL reference value (hereinafter, SCL_{base}: a first reference value) and the SCR reference value (SCR_{base}: a second reference value). The SCL_{base} is the SCL value in the case where the activity state is the quiet state and emotions (the psychological state) are in the physiologically quiet state. Similarly, SCR_{base} is the SCR value in the case where the activity state is the quiet state and emotions (the psychological state) are in the physiologically quiet state.

The reference value storage unit 37 is configured to be capable of determining, on the basis of the SCR signal, whether emotions are in the physiologically quiet state or physiologically non-quiet state. Note that the reference value storage unit 37 executes, in the case where the situation in the activity state is the quiet state regarding the determination result input by the activity state analysis unit 38, this determination of the physiologically quiet state/non-quiet state. Meanwhile, the reference value storage unit 37 typically does not execute, in the case where the situation in the activity state is the activity state and the quasi-activity state, the determination of the physiologically quiet state/non-quiet state.

Regarding the determination of the physiologically quiet state/non-quiet state, the reference value storage unit 37 analyzes the SCR signal to obtain the occurrence frequency of SCR. Then, the reference value storage unit 37 determines, in the case where the occurrence frequency of SCR is less than a threshold value, that emotions are in the physiologically quiet state. Meanwhile, the reference value storage unit 37 determines, in the case where the occurrence frequency of SCR is the threshold value or more, that emotions are in the physiologically non-quiet state.

Note that SCR used in the determination of the physiologically quiet state/non-quiet state may be SCR before correction or SCR after correction. Further, the subject that executes the determination of the physiologically quiet state/non-quiet state may be the activity state analysis unit 38 instead of the reference value storage unit 37.

In the description here, although the case where the SCR signal is used in the determination of the physiologically quiet state/non-quiet state has been described, the SCL signal may be used instead of the SCR signal in this determination.

In this case, for example, the reference value storage unit 37 obtains the average value of the SCL signal in a predetermined time. Then, the reference value storage unit 37 determines, in the case where the average value of the SCL signal is less than a predetermined threshold value, that emotions are in the physiologically quiet state and determines, in the case where the average value of the SCL signal is the threshold value or more, that emotions are in the physiologically non-quiet state.

Note that in the determination of the physiologically quiet state/non-quiet state, both the SCR signal and the SCL signal may be used. Further, the reference value storage unit 37 may perform the determination of the physiologically quiet state/non-quiet state on the basis of a skin conductance signal (before separation). Typically, the reference value storage unit 37 only needs to be configured to be capable of determining, on the basis of the signal (the SCR signal, the SCL signal, the skin conductance signal itself) regarding a skin conductance signal (perspiration signal), whether emotions are in the physiologically quiet or the physiologically non-quiet state.

In the case where the activity state is the quiet state and the emotions are in the physiologically quiet state, the reference value storage unit 37 stores the SCL value and the SCR value at that time. Meanwhile, in other cases, i.e., in the case where the situation in the activity state is the activity state and the quasi-activity state, and in the case where the situation in the activity state is the quiet state and the emotions are in the physiologically non-quiet state, the reference value storage unit 37 does not store the SCL value and the SCR value at that time.

The reference value storage unit 37 calculates the average value of SCL values and the average value of SCL values in the case where the activity state is the quiet state and the emotions are in the physiologically quiet state. The reference value storage unit 37 stores the average value of SCL as SCL_{base} and the average value of SCR as SCR_{base}.

The reference value storage unit 37 is configured to be capable of outputting information of SCL_{base} and SCR_{base} to the difference extraction unit 36.

(Difference Extraction Unit 36)

An SCL signal and an SCR signal are input from the SCL/SCR separation unit 35 to the difference extraction unit 36. Further, information of SCL_{base} and SCR_{base} is input from the reference value storage unit 37 to the difference extraction unit 36.

The difference extraction unit 36 is configured to be capable of extracting a difference between the SCL value and SCL_{base} and a difference between the SCR value and SCR_{base}. The difference extraction unit 36 subtracts SCL_{base} from the SCL value to calculate dSCL that is a difference. Further, the difference extraction unit 36 subtracts SCR_{base} from the SCR value to calculate dSCR that is a difference.

The difference extraction unit 36 is configured to be capable of outputting the calculated dSCL value and dSCR value to the correction processing unit 39.

(Correction Processing Unit 39)

A dSCL value and a dSCR value are input from the difference extraction unit 36 to the correction processing unit 39. The correction processing unit 39 is configured to be capable of correcting dSCR on the basis of dSCL. Typically, the correction processing unit 39 is configured to be capable of correcting dSCR by a gain relating to dSCL, and the gain is set to a value that monotonically decreases with respect to dSCL. Note that in the example here, a case where dSCR is corrected on the basis of dSCL will be described, but SCR itself may be corrected on the basis of SCL itself.

Specifically, the correction processing unit 39 applies a look-up table to the input dSCL value to obtain a gain that is a function of dSCL. Note that gein=f(dSCL). Then, the correction processing unit 39 multiplies dSCR by the gain to obtain SCR after correction (dSCR′). Note that dSCR′=dSCR×gein.

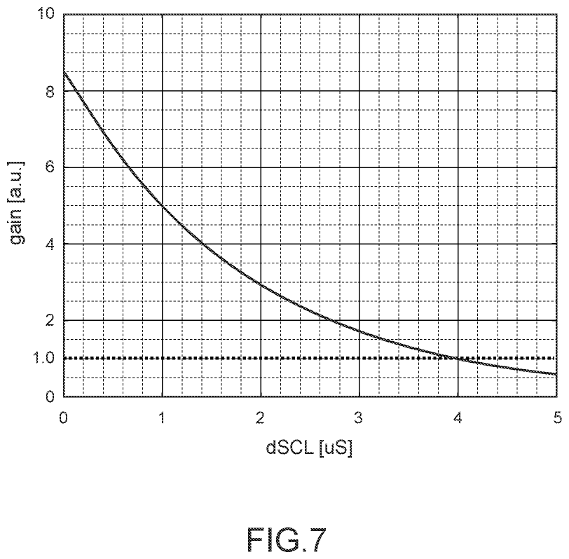

The gain is a value that monotonically decreases with respect to the dSCL value that is an input variable. FIG. 7 is a diagram showing an example of the relationship between the gain and dSCL. In the example shown in FIG. 7, an example in the case where gein=f(dSCL)=b×exp(−dSCL/a) is shown. Note that the gain only needs to be a value that monotonically decreases with respect to the dSCL value and is not limited to this example.

The corrected SCR signal is used for analyzing emotions (the psychological state) of a living body. For example, emotions of a user such as the tense state, the relaxed state, the joyful state, and the pessimistic state are determined on the basis of the corrected SCR signal. Information of emotions can be used for various purposes.

For example, the difficulty level of the game may be changed in accordance with the tense state or the relaxed state of the user. Further, emotions of the user may be analyzed when the user is playing golf, and may be used for determining whether or not he/she is swinging in the relaxed state. Further, emotions of the user may be analyzed when the user is doing yoga to determine whether or not yoga leads to improvement in the mental state.

### Experimental Example

Next, the psychological experimental task performed on a subject and the relationship between SCL and SCR at that time will be described.

(SCL and SCR Values with “Finger”)

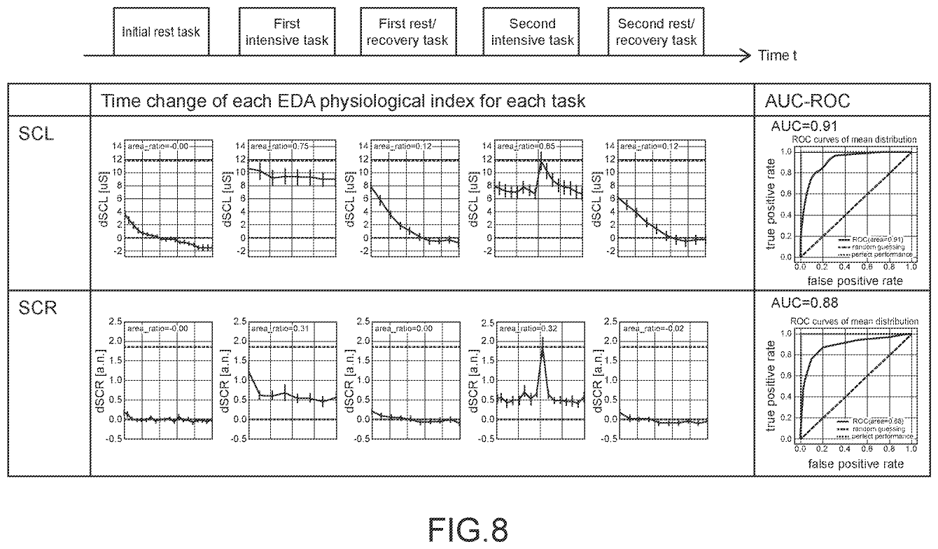

FIG. 8 is a diagram showing the relationship between SCL and SCR (dSCL and dSCR) with a finger for each experimental task. In this experimental task, SCL and SCR (dSCL and dSCR) are measured for each of “fingers” of 19 subjects. In this experimental task, tasks were performed in the order of an initial rest task, a first intensive task, a first rest/recovery task, a second intensive task, and a second rest/recovery.

Note that hereinafter, the initial rest task, the first rest/recovery task, and the second rest/recovery task will be collectively referred to simply as the rest task. Further, the first intensive task and the second intensive task will be collectively referred to simply as the intensive task.

In the initial rest task, the subjects were asked to rest for a predetermined time period (a few minutes to a dozen minutes). The graph shown in FIG. 8 is a graph in which the average value of SCL values and the average value of SCR values for 19 subjects in the initial rest task are set to zero and the average value of SCL values and the average value of SCR values for 19 subjects are normalized. Note that the average value of SCL values and the average value of SCR values in the initial rest task respectively correspond to SCL_{base} and SCR_{base} Further, in FIG. 8, since this value is used as a reference value (zero), the vertical axis corresponds to dSCL and dSCR.

In the initial rest task, SCL and SCR (dSCL and dSCR) have remained relatively stable near 0.

In the first intensive task, a psychological load task for inducing concentration was performed on the subjects for a predetermined time period (a few minutes to a dozen minutes).

In this first intensive task, SCL and SCR (dSCL and dSCR) rises sharply at the time of switching from the initial rest task to take a high value and then remains stable at a high value.

In the first rest/recovery task, the subjects were asked to rest for recovery for a predetermined time period (a few minutes to a dozen minutes). In this first rest/recovery task, SCL (dSCL) gradually declines to approach 0 and then remains stable near 0. Meanwhile, SCR (dSCR) declines more quickly than SCL at the time of switching from the first intensive task to approach 0 and then remains stable near 0.

In the second intensive task, a psychological load task for inducing concentration was performed on the subjects for a predetermined time period (a few minutes to a dozen minutes). Note that in this second intensive task, a task different from that in the first intensive task was performed. In the second intensive task, a task with a relatively small psychological load (hereinafter, the first half task) was performed in the first half, and a task with a relatively large psychological load (hereinafter, the second half task) was performed in the second half.

In the second intensive task, SCL (dSCL) rises sharply at the time of switching from the first rest/recovery task to take a high value and then remains stable at the high value during the first half task. Further, SCL (dSCL) takes a higher value for a moment at the time of switching between the first half task and the second half task and then gradually declines to approach the original high value.

Further, in the second intensive task, SCR (dSCR) rises sharply at the time of switching from the first rest/recovery task to take a high value and then remains stable at the high value during the first half task. Further, SCR (dSCR) takes a higher value for a moment at the time of switching between the first half task and the second half task and then approaches the original high value more quickly than SCL to remain near the high value.

In the second rest/recovery task, the subjects were asked to rest for recovery for a predetermined time period (a few minutes to a dozen minutes). In this second rest/recovery task, SCL (dSCL) gradually declines to approach 0 and then remains stable near 0. Meanwhile, SCR (dSCR) declines more quickly than SCL at the time of switching from the second intensive task to approach 0 and then remains stable near 0.

As is clear from FIG. 8, SCL and SCR (dSCL and dSCR) tend to have relatively low values in the rest task including the initial rest task, the first rest/recovery task, and the second rest/recovery task. On the contrary, SCL and SCR (dSCL and dSCR) tend to take relatively high values in the intensive task including the first intensive task and the second intensive task.

In the upper right of FIG. 8, the degree of separation between SCL (dSCL) in the rest task and SCL (dSCL) in the intensive task is represented by AUC (Area Under the Curve) of the ROC curve (ROC: Receiver Operating Characteristic). Further, in the lower right of FIG. 8, the degree of separation between SCR (dSCR) in the rest task and SCR (dSCR) in the intensive task is represented by AUC of the ROC curve.

The value of AUC, which is the lower area of the ROC curve, indicates the degree of separation between the physiological index (SCL and SCR) in the rest task and the physiological index in the intensive task, i.e., how much these can be separated and identified.

The AUC value takes a value from 0.5 to 1. Complete separation is possible in the case where the AUC value is 1, and conversely, completely-random separation is performed in the case where the AUC value is 0.5.

With reference to the upper right of FIG. 8, in SCL (dSCL), the degree of separation between the rest task and the intensive task satisfies the relationship of AUC=0.91. Further, with reference to the lower right of FIG. 8, in SCR (dSCR), the degree of separation between the rest task and the intensive task satisfies the relationship of AUC=0.88.

That is, in the case where a “finger” that is a location with many sweat glands is a measurement target, both SCL and SCR show a high value of the degree of separation between the rest task and the intensive task. That is, this means that in the case where the measurement target is a finger, the difference in SCL and SCR between the rest task and the intensive task is large.

Note that the higher the AUC value and the higher the degree of separation, the more accurately emotions (the psychological state) of a living body can be inferred.

(SCL and SCR Values with “Wrist”)

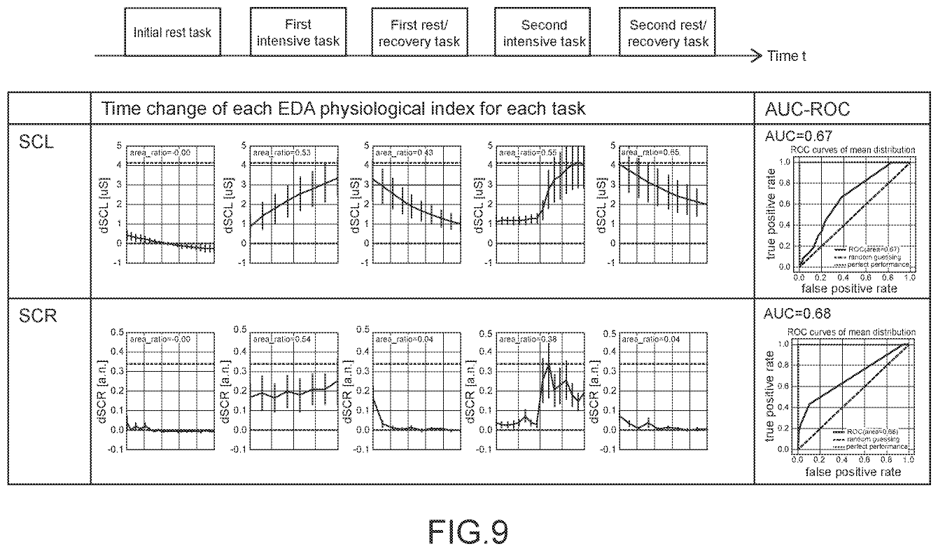

FIG. 9 is a diagram showing the relationship between SCL and SCR (dSCL and dSCR) with the wrist for each experimental task. In the example shown in FIG. 9, the measurement target has been not a finger but a “wrist”. The method of measuring SCL and SCR is the same as that in the case described in FIG. 8.

In FIG. 9, the scale of the vertical axis in SCL and SCR (dSCL and dSCR) is different from that in FIG. 8. That is, while the scale of the vertical axis in SCL (dSCL) is 14 [μS] in FIG. 8, the scale of the vertical axis in SCL (dSCL) is 5 [μS] in FIG. 9. Further, while the scale in the vertical axis of SCR (dSCR) is 2.5 in FIG. 8, the scale in the vertical axis of SCL (dSCL) is 0.5 in FIG. 9.

As is clear from the comparison of FIG. 8 and FIG. 9, the SCL and SCR values of the wrist with a few sweat gland are clearly lower than the SCL and SCR values of the finger with many sweat glands.

The graph of FIG. 9 will be specifically described. In the initial rest task, SCL and SCR (dSCL and dSCR) remain relatively stable near 0.

In the first intensive task, SCL (dSCL) gradually rises from the time of switching from the initial rest task. Further, SCR (dSCR) rises at the time of switching from the initial rest task and then remains stable at the value.

In the first rest task, SCL (dSCL) gradually declines from the time of switching from the first intensive task. Meanwhile, SCR (dSCR) declines more quickly than SCL at the time of switching from the first intensive task to approach 0 and then remains stable near 0.

In the second intensive task, SCL (dSCL) remains stable at a slightly lower value during the first half task. Further, SCL (dSCL) gradually rises from the time of switching between the first half task and the second half task.

Further, in the second intensive task, SCR (dSCR) slightly rises at the time of switching from the first rest/recovery task but remains at a low value during the first half task. Further, SCR (dSCR) rises sharply at the time of switching between the first half task and the second half task and then gradually declines.

In the second rest/recovery task, SCL (dSCL) gradually declines from the time of switching from the second intensive task. Meanwhile, SCR (dSCR) declines more quickly than SCL at the time of switching from the second intensive task to approach 0 and then remains stable near 0.

Also in FIG. 9, SCL and SCR (dSCL and dSCR) tend to take relative low values in the rest task and take relatively high values in the intensive task. However, in the case of FIG. 9 (wrist), this tendency is clearly smaller than that in FIG. 8 (finger).

In the upper right of FIG. 9, the degree of separation between SCL (dSCL) in the rest task and SCL (dSCL) in the intensive task is represented by AUC of the ROC curve. Further, in the lower right of FIG. 9, the degree of separation between SCR (dSCR) in the rest task and SCR (dSCR) in the intensive task is represented by AUC of the ROC curve.

With reference to the upper right of FIG. 9, in SCL (dSCL), the degree of separation between the rest task and the intensive task satisfies the relationship of AUC=0.67. Further, with reference to the lower right of FIG. 9, in SCR (dSCR), the degree of separation between the rest task and the intensive task satisfies the relationship of AUC=0.68.

That is, in the case where the “wrist” that is a location with a few sweat gland is a measurement target, both SCL and SCR show a low value of the degree of separation between the rest task and the intensive task. That is, this means that in the case where the measurement target is the wrist, there is not much difference in SCL and SCR between the rest task and the intensive task. Note that when the AUC value is low and the degree of separation is low, the accuracy for inferring emotions (the psychological state) of a living body decreases.

Here, regarding the SCR (dSCR) value of FIG. 8 (finger) and FIG. 9 (wrist), the first half period of the second intensive task is focused on. During this period, a psychological load task is performed and the subject is in the mentally tense state.

In FIG. 8 (finger), during this first half period, SCR (dSCR) remains stable at a relatively high value, i.e., a value having a large difference from that value at the time of the rest task. Meanwhile, in the case of FIG. 9 (wrist), during this first half period, SCR (dSCR) remains at a relatively low value, i.e., a value having not much difference from the value at the time of the rest task.

As described above, since SCR (dSCR) takes a value having not much difference from the value at the time of the rest task in the case of FIG. 9 (wrist), there is a possibility that even if the subject is in the tense state in the first half period of the second intensive task, he/she is erroneously determined to be not in the tense state.

For this reason, in the present technology, the SCR (dSCL) value is corrected on the basis of the SCL (dSCL) value.

(Comparison of SCR without Correction and SCR with Correction with “Wrist”)

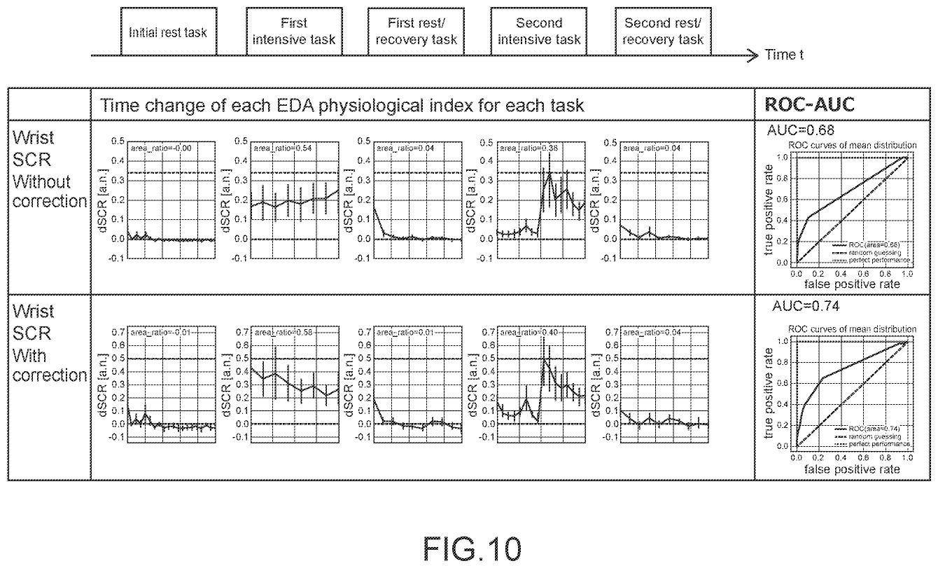

Next, comparison of SCR without correction (before correction) and SCR with correction (after correction) in the case where the measurement target is the wrist will be described. FIG. 10 is a diagram comparing the relationship between SCR without correction and SCR with correction in the case where the measurement target is the wrist.

On the upper side of FIG. 10, SCR (dSCR) without correction (before correction) with the wrist is shown. This SCR (dSCR) shown on the upper side of FIG. 10 is the same as SCR (dSCR) shown on the lower side of FIG. 9. Further, on the lower side of FIG. 10, SCR (dSCR) with correction (after correction) with the wrist is shown.

SCR (dSCR) with correction (after correction) on the lower side of FIG. 10 is a value obtained by multiplying SCR (dSCR) without correction (before correction) on the upper side of FIG. 10 by a gain (see FIG. 7). Note that as described above, the gain is a value that monotonically decreases with respect to the SCL (dSCL) value.

In the description of FIG. 10, SCL shown on the upper side of FIG. 9 is also referred to (because SCL is involved in the correction of SCR). Note that in FIG. 10, the scale of the vertical axis slightly differs between the case without correction on the upper side and the case with correction on the lower side. Specifically, while the scale of the vertical axis in SCR without correction is 0.5, the scale of the vertical axis in SCR with correction is 0.7.

First, the initial rest task will be described. As shown in the upper side of FIG. 9, in the initial rest task, the SCL (dSCL) value remains stable near 0 (the average value of SCL values in the initial rest task corresponds to SCL_{base}) The gain to be multiplied by SCR (dSCR) is a value that monotonically decreases with respect to the SCL (dSCL) value (see FIG. 7). Therefore, in the initial rest task, a relatively high value is used as the gain.

With reference to the lower side of FIG. 9 and the upper side of FIG. 10, in the initial rest task, the SCR (dSCR) value remains stable near 0 (because the average value of SCR values in the initial rest task corresponds to SCR_{base}) Therefore, in the initial rest task, although a relatively high value is used as the gain, the SCR (dSCR) value is a value near 0 in the first place. Therefore, as can be seen from the comparison before and after correction on the upper side and lower side of FIG. 10, in the initial rest task, SCR (dSCR) remains at a low value and does not change much even if the gain is multiplied for correction.

Next, the first intensive task will be described. As shown in the upper side of FIG. 9, in the first intensive task, the SCL (dSCL) value gradually rises while taking a relatively high value. Therefore, in the first intensive task, a relatively low value is used as the gain. Further, in the first intensive task, since the SCL (dSCL) value gradually rises, the gain gradually decreases.

Note that in the example here, as shown in FIG. 7, in the case where SCL (dSCL) is 4 [μS] or less, a value of 1 or more is used as the gain. As shown in the upper side of FIG. 9, in the first intensive task, the SCL (dSCL) value is 4 [μS] or less at any time. Therefore, in the first intensive task, although a relatively low value is used as the gain, the value exceeds 1 and the SCR (dSCL) value after correction rises not a little than the original value. The same applies to other tasks.

With reference to the lower side of FIG. 9 and the upper side of FIG. 10, in the first intensive task, the SCR (dSCR) value takes a relatively high value (gradual rise tendency). In the first intensive task, this relatively high SCR (dSCR) value is multiplied by a gain of a relatively low value (1 or more) to perform correction. As can be seen from the comparison between the upper side and the lower side of FIG. 10, in the first intensive task, when SCR (dSCR) is multiplied by the gain to perform correction, it reaches a high value as a whole than that before correction.

Next, the first rest/recovery task will be described. As shown in the upper side of FIG. 9, in the first rest/recovery task, the SCL (dSCL) value gradually declines while taking a relatively high value. Therefore, in the first rest/recovery task, a relatively low value is used as the gain (1 or more). Further, in the first rest/recovery task, since the SCL (dSCL) value gradually declines, the gain gradually increases.

With reference to the lower side of FIG. 9 and the upper side of FIG. 10, in the first rest/recovery task, the SCR (dSCR) value declines first and then remains stable at a value near 0.

The first period (several tens of seconds) in the first rest/recovery task will be described. SCR before correction in the first rest/recovery task takes a slightly higher value until it declines to near 0 in the first period. Meanwhile, in the first period, the corresponding SCL (dSCL) value is high and therefore, the value of the gain to be multiplied by this SCR is small. For this reason, as can be seen from the comparison between the upper side and the lower side of FIG. 10, in the first rest/recovery task, SCR (dSCR) does not much change even if the gain is multiplied to perform correction in the first period.

The rest of the period after the first period has elapsed in the first rest/recovery task will be described. SCR before correction in the first rest/recovery task remains stable at a value near 0 in the rest of the period. This SCR (dSCR) taking a value near 0 is multiplied by the gain of a relatively low value to correct SCR. Therefore, as can be seen from the comparison between the upper side and the lower side of FIG. 10, SCR (dSCR) remains at a low value and does not much change even if correction is performed in the rest of the period in the first rest/recovery task.

Next, the second intensive task will be described. As shown in the upper side of FIG. 9, in the second intensive task, SCL (dSCL) remains stable while taking a slightly lower value in the first half. Further, SCL (dSCL) gradually rises from the time of switching between the first half task and the second half task.

Therefore, in the first half of the second intensive task, a relatively high value is used as the gain. Further, in the first half of the second intensive task, the gain gradually decreases from a relatively high value to a relatively low value.

With reference to the lower side of FIG. 9 and the upper side of FIG. 10, in the second intensive task, SCR (dSCR) slightly rises at the time of switching from the first rest/recovery task but remains at a low value in the first half. Further, SCR (dSCR) rises sharply at the time of switching between the first half task and the second half task and then gradually declines.

The first half period in the second intensive task will be described. SCR (dSCR) before correction in the second intensive task remains at a low value in the first half period but the corresponding SCL (dSCL) value is relatively low. Therefore, this SCR (dSCR) is multiplied by the gain of a relatively high value to perform correction. Therefore, as can be seen from the comparison between the upper side and the lower side of FIG. 10, in the second intensive task, when SCR (dSCR) is multiplied by the gain to perform correction in the first half period, SCR (dSCR) takes a high value with respect to the original value.

The period of the second half in the second intensive task will be described. SCR (dSCR) before correction in the second intensive task rises sharply at the time of switching between the first half task and the second half task and then gradually declines. Meanwhile, the corresponding SCL (dSCR) rises slower than SCR (dSCR) and gradually rises even after SCR has declined. Therefore, in the period of the second half in the second intensive task, SCR that gradually declines is multiplied by the gain that gradually decreases (relatively high at first) to perform correction.

Therefore, as can be seen from the comparison between the upper side and the lower side of FIG. 10, in the second intensive task, when SCR (dSCR) is multiplied by the gain to perform correction in the second half, the difference between the top and bottom of SCR (dSCR) is slightly larger than that of the original SCR.

Since the second rest/recovery task is substantially the same as the first rest/recovery task, description thereof will be omitted.

With reference to the upper right of FIG. 10, in SCR (dSCR) without correction (before correction), the degree of separation between the rest task and the intensive task satisfies the relationship of AUC=0.67. Meanwhile, with reference to the lower right of FIG. 9, in SCR (dSCR) with correction (after correction), the degree of separation between the rest task and the intensive task satisfies the relationship of AUC=0.74.

That is, in this embodiment, by correcting SCR (dSCR) as described above, the degree of separation between the rest task and the intensive task can be improved. As described above, by improving the degree of separation, it is possible to obtain an SCR accurately representing emotions of a living body and improve the accuracy for inferring the emotions (the psychological state) of the living body.

In particular, in the example here, the SCR (dSCR) value in the first half period in the second intensive task is appropriately high enough to be separatable from the SCR (dSCR) value in the rest task. For this reason, it is possible to prevent the subject from being erroneously determined to be not in the tense state although he/she is in the tense state.

Here, a case where the gain multiplied by SCR (dSCR) monotonically increases with respect to SCL (dSCL) will be described. In this case, the first period (several tens of seconds) in the first rest/recovery task and the second rest/recovery task will be focused on.

With reference to the lower side of FIG. 9 and the upper side of FIG. 10, in the first period in the rest/recovery task (general term for the first rest/recovery task and the second rest/recovery task), SCR (dSCR) before correction takes a slightly higher value until it declines to near 0. With reference to the upper side of FIG. 9, in the first period in the rest/recovery task, the SCL (dSCL) value takes a high value although it has begun to decline. In this case, if the gain monotonically increases with respect to SCL (dSCL), a relatively high value is used as the gain.

Therefore, in the first period of the rest/recovery task, SCR (dSCR) is corrected when SCR (dSCR) of a relatively high value is multiplied by the gain of a relatively high value. In this case, in the first period of the rest/recovery task, SCR is corrected as a high value and output, and there is a possibility that it is determined to be in the tense state, for example. Meanwhile, the first period of the rest/recovery task is originally the start period of the relaxed state and is not such a period of being in the tense state.

As described above, a case where it is not appropriate when the gain monotonically increases with respect to SCL (dSCL) is assumed. Meanwhile, in this embodiment, the gain monotonically decreases with respect to SCL (dSCL). Therefore, as described above, even in the case where SCR (dSCR) is multiplied by the gain to perform correction in the first period in the rest/recovery task, SCR (dSCR) remains at a low value and does not change much. Therefore, it is possible to prevent the above-mentioned erroneous determination.

That is, in this embodiment, since the gain monotonically decreases with respect to SCL (dSCL), for example, it is possible to correct SCR that is originally desired to be high, such as that in the first half period of the second intensive task, to be higher and cause SCR that is desired to be a low value, such as that in the first period in the rest/recovery task, to remain at a low value. As described above, in this embodiment, since the gain monotonically decreases with respect to SCL (dSCL), it is possible to appropriately correct SCR to improve the detection sensitivity of SCR.

<Operations, Etc.>

As described above, in this embodiment, a skin conductance signal is separated into SCL (dSCL) and SCR (dSCL), and SCR (dSCL) is corrected on the basis of SCL (dSCL).

As a result, it is possible to obtain SCR that accurately represents the emotional reaction of a living body and is corrected so that the detection sensitivity is high, and infer emotions (the psychological state) of the living body using SCR after correction. In particular, in this embodiment, for example, even in the case where a skin conductance signal is detected in a part with a few sweat gland such as the wrist, it is possible to appropriately correct SCR and accurately infer emotions of a living body.

Further, in this embodiment, even if a skin conductance signal is detected in a part with a few sweat gland, such as the wrist, it is possible to infer emotions in real time without delay. As a result, for example, it is possible to use information of emotions of a user in real time in various applications such as games and the use of the information is expected to expand in various applications.

Further, in this embodiment, the gain to be multiplied by SCR (dSCR) is a value relating to SCL (dSCL), and in particular, the gain monotonically decreases with respect to SCL (dSCL). As a result, it is possible to appropriately correct SCR (dSCR).

Further, in this embodiment, dSCL that is a value of SCL with respect to SCL_{base} and dSCR that is a value of SCR with respect to SCR_{base} are used. As a result, for example, it is possible to absorb the individual difference between SCL and SCR.

Further, in this embodiment, SCL_{base} is SCL in the case where the activity state is the quiet state and emotions are in the physiologically quiet state. Further, SCR_{base} is SCR in the case where the activity state is the quiet state and emotions are in the physiologically quiet state. As a result, it is possible to use appropriate SCL_{base} and SCR_{base} as reference values.

Further, in this embodiment, whether the situation in the activity state is the non-quiet state or the quiet state is determined on the basis of at least one of the inertial signal and the pressure signal. As a result, it is possible to appropriately determine the non-quiet state/quiet state in the activity state.

Further, in this embodiment, whether emotions are in the physiologically non-quiet state or quiet state is determined on the basis of a signal relating to a skin conductance signal (an SCL signal, an SCR signal (before correction and after correction), the skin conductance signal itself). As a result, it is possible to appropriately determine the physiologically non-quiet state/quiet state in emotions.

### Various Modified Examples

In the above description, the case where processes such as separation of a skin conductance signal into SCR/SCR and correction of SCR are executed by the control unit 1 of the wearable device 10 has been described. Meanwhile, the above-mentioned processes may be executed by, for example, an external device such as a mobile phone (including a smartphone), a PC (a tablet PC, a laptop PC, a desktop PC, or the like), or a server apparatus on the network. In this case, the wearable device 10 transmits, to an external device, information such as a skin conductance signal, an inertial signal, and a pressure signal as necessary. The external device executes the above-mentioned processes on the basis of the respective received information. Note that part of the above-mentioned processes may be executed by the wearable device 10 and the other part may be executed by an external device.

Although a wristwatch type (wristband type) wearable device 10 has been described as an example of the information processing apparatus in the above description, the information processing apparatus is not limited thereto. For example, the information processing apparatus may be various other wearable devices 10 such as a glove type, a ring type, a headband type, a glasses type, a hat type, an accessory type, a clothing type, and a shoe type (the number of sweat glands at the contact position does not matter).

Further, the information processing apparatus may be an apparatus other than the wearable device 10. For example, the information processing apparatus may be provided on the surface or inside of an object to be in contact with a user. Examples thereof in this case include a mobile phone (including a smartphone), a PC, a mouse, a keyboard, a handle, a lever, a camera, exercise equipment (a golf club, a tennis racket, etc.), and a writing utensil. Typically, the information processing apparatus may be of any form as long as it can come into contact with the skin of a human (or animal) (the number of sweat glands at the contact position does not matter). Note that the information processing apparatus may be the external device (a mobile phone, a PC, a server apparatus, or the like) as described above.

The present technology may also take the following configurations.

(1) An information processing apparatus, including:

a control unit that separates a perspiration signal into a first fluctuation component and a second fluctuation component and corrects the second fluctuation component on a basis of the first fluctuation component.

(2) The information processing apparatus according to (1) above, in which

the control unit corrects the second fluctuation component by a gain relating to the first fluctuation component.

(3) The information processing apparatus according to (2) above, in which

the gain is a value that monotonically decreases with respect to a value of the first fluctuation component.

(4) The information processing apparatus according to (3) above, in which

the gain is a value that monotonically decreases with respect to a value of the first fluctuation component with respect to a first reference value.

(5) The information processing apparatus according to (4) above, in which

the control unit determines whether or not emotions are in a physiologically quiet state on a basis of a signal relating to the perspiration signal, and

the first reference value is the first fluctuation component in a case where the emotions are in the physiologically quiet state.

(6) The information processing apparatus according to (5) above, in which

the control unit determines whether or not an activity state is a quiet state on a basis of at least one of a body motion signal based on a body motion change or a pressure signal based on a pressure change with skin, and

the first reference value is the first fluctuation component in a case where the activity state is the quiet state and the emotions are in the physiologically quiet state.

(7) The information processing apparatus according to any one of (1) to (6) above, in which

the control unit corrects a value of the second fluctuation component with respect to a second reference value.

(8) The information processing apparatus according to (7) above, in which

the control unit determines whether or not the emotions are in the physiologically quiet state on a basis of a signal relating to the perspiration signal, and

the second reference value is the second fluctuation component in a case where the emotions are in the physiologically quiet state.

(9) The information processing apparatus according to (8) above, in which

the control unit determines whether or not the activity state is the quiet state on a basis of at least one of the body motion signal based on the body motion change or the pressure signal based on the pressure change with the skin, and

the second reference value is the second fluctuation component in a case where the activity state is the quiet state and the emotions are in the physiologically quiet state.

(10) The information processing apparatus according to any one of (1) to (9) above, in which

the first fluctuation component is a baseline fluctuation component of the perspiration signal.

(11) The information processing apparatus according to any one of (1) to (10) above, in which

the second fluctuation component is an instantaneous fluctuation component of the perspiration signal.

(12) An information processing method, including:

separating a perspiration signal into a first fluctuation component and a second fluctuation component; and

correcting the second fluctuation component on a basis of the first fluctuation component.

(13) A program according that causes a computer to execute the following processing of:

separating a perspiration signal into a first fluctuation component and a second fluctuation component; and

correcting the second fluctuation component on a basis of the first fluctuation component.

### REFERENCE SIGNS LIST

1 control unit
2 perspiration sensor
3 inertia sensor
4 pressure sensor
10 wearable device
35 SCL/SCR separation unit
36 difference extraction unit
37 reference value storage unit
38 activity state analysis unit
39 correction processing unit

1. An information processing apparatus, comprising:
a control unit that separates a perspiration signal into a first fluctuation component and a second fluctuation component and corrects the second fluctuation component on a basis of the first fluctuation component.

2. The information processing apparatus according to claim 1, wherein
the control unit corrects the second fluctuation component by a gain relating to the first fluctuation component.

3. The information processing apparatus according to claim 2, wherein
the gain is a value that monotonically decreases with respect to a value of the first fluctuation component.

4. The information processing apparatus according to claim 3, wherein
the gain is a value that monotonically decreases with respect to a value of the first fluctuation component with respect to a first reference value.

5. The information processing apparatus according to claim 4, wherein
the control unit determines whether or not emotions are in a physiologically quiet state on a basis of a signal relating to the perspiration signal, and
the first reference value is the first fluctuation component in a case where the emotions are in the physiologically quiet state.

6. The information processing apparatus according to claim 5, wherein
the control unit determines whether or not an activity state is a quiet state on a basis of at least one of a body motion signal based on a body motion change or a pressure signal based on a pressure change with skin, and
the first reference value is the first fluctuation component in a case where the activity state is the quiet state and the emotions are in the physiologically quiet state.

7. The information processing apparatus according to claim 1, wherein
the control unit corrects a value of the second fluctuation component with respect to a second reference value.

8. The information processing apparatus according to claim 7, wherein
the control unit determines whether or not the emotions are in the physiologically quiet state on a basis of a signal relating to the perspiration signal, and
the second reference value is the second fluctuation component in a case where the emotions are in the physiologically quiet state.

9. The information processing apparatus according to claim 8, wherein
the control unit determines whether or not the activity state is the quiet state on a basis of at least one of the body motion signal based on the body motion change or the pressure signal based on the pressure change with the skin, and
the second reference value is the second fluctuation component in a case where the activity state is the quiet state and the emotions are in the physiologically quiet state.

10. The information processing apparatus according to claim 1, wherein
the first fluctuation component is a baseline fluctuation component of the perspiration signal.

11. The information processing apparatus according to claim 1, wherein
the second fluctuation component is an instantaneous fluctuation component of the perspiration signal.

12. An information processing method, comprising:
separating a perspiration signal into a first fluctuation component and a second fluctuation component; and
correcting the second fluctuation component on a basis of the first fluctuation component.

13. A program according that causes a computer to execute the following processing of:
separating a perspiration signal into a first fluctuation component and a second fluctuation component; and
correcting the second fluctuation component on a basis of the first fluctuation component.
